(19) United States
(12) Patent Application Publication
(10) Pub. No.: US 2026/0354858 A1
(43) Pub. Date: Nov. 12, 2026

(54) SYSTEMS AND METHOD AND DEVICES FOR TARGETED NEEDLE PLACEMENT USING REAL-TIME ULTRASOUND GUIDANCE

(71) Applicant: Zachary D. Walton, Gloucester, MA (US)

(72) Inventor: Zachary D. Walton, Gloucester, MA (US)

(21) Appl. No.: 19/665,327

(22) Filed: May 1, 2026

Related U.S. Application Data
(60) Provisional application No. 63/800,532, filed on May 6, 2025.
(60) Provisional application No. 63/800,541, filed on May 6, 2025.

(51) Int. Cl. A61B 17/34

(52) U.S. Cl. CPC A61B 17/3403; A61B 2017/3413

(57) ABSTRACT

Aspects of the present disclosure describe medical device placement, including devices, systems and methods for placing other medical devices, such as needles, using a guide on a sterile ultrasound sheath.

### CROSS-REFERENCE TO RELATED APPLICATIONS

The present application claims the benefit of U.S. Provisional Patent Application Ser. No. 63/800,532, filed May 6, 2025 and U.S. Provisional Patent Application Ser. No. 63/800,541, filed May 6, 2025, the entire contents of which are specifically incorporated herein by reference.

### FIELD

The present technology is generally related to medical device placement, for example, needles, etc., using an assistive medical device for such placement with real-time ultrasound guidance.

### BACKGROUND

Exemplary procedures involving needle insertion into a patient's body, particularly alongside real-time ultrasound, generally includes, without limitation, anesthesia (e.g., nerve blocks, pain procedures) and interventional radiology (e.g., cyst drainage, biopsy).

The present disclosure recognizes that use of needle placement in conjunction with real-time ultrasound guidance can involve complex or bulky components that can be difficult to coordinate for accurate placement and that typically require multiple operators to effectively provide placement by attempting to maintain proper visualization of the image as the needle is advanced.

Maintaining visualization during needle advancement can also be particularly challenging when the patient is obese. In this case, obtaining the image requires pushing several centimeters into the skin of the patient. The probe is touching the skin at the end of a tunnel; and there is no flat area of skin adjacent to the probe suitable for a needle entry site. Often an assistant is required to hold back the skin to allow access to the needle entry site. Even with assistance, this process can be difficult because the process of moving the skin away from the probe can cause loss of target visualization.

Accordingly, the present disclosure recognizes that there is a need in the art for improvements in ease, reliability, and repeatability of medical device placement. This disclosure introduces devices and methods for using ultrasound for real-time guidance of medical device placement for needle placement that overcomes the above-described and other challenges and problems in the art.

### SUMMARY

The techniques of this disclosure generally relate to medical device placement, including devices, systems and methods for placing needles, using a guide on a sterile ultrasound sheath.

Exemplary embodiments describe a shell that at least partially houses the ultrasound probe. In exemplary embodiments, this shell is a rigid, sterile single use shell. Connected to or integral to the shell is a flange that moves the patient's skin away from the probe as it presses against the patient's skin. At the connection of the flange to the distal point of the shell, there are plural passageways for needles to be advanced at one or more fixed angles and/or at a continuum of angles, in either the in-plane or out-of-plane configuration relative to the ultrasound probe positioning.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a front elevation view of a first exemplary embodiment of a rigid ultrasound probe shell, in accordance with exemplary aspects of the present disclosure;

FIG. 2 is a bottom elevation view of the distal end of the rigid probe shell of FIG. 1, in accordance with exemplary aspects of the present disclosure;

FIG. 3 is a, in-plane cross-sectional view of the exemplary shell of FIG. 1, in accordance with exemplary aspects of the present disclosure;

FIG. 4 is an out-of-plane cross sectional view of the exemplary shell of FIG. 1, in accordance with exemplary aspects of the present disclosure;

FIG. 5 is a rear elevation view of another exemplary shell embodiment, in accordance with exemplary aspects of the present disclosure;

FIG. 6 is a front elevation view of the exemplary embodiment of FIG. 5, in accordance with exemplary aspects of the present disclosure;

FIG. 7 is a top perspective view of the exemplary embodiment of FIG. 5, in accordance with exemplary aspects of the present disclosure; and

FIG. 8 is a bottom elevation view of the exemplary embodiment of FIG. 5, in accordance with exemplary aspects of the present disclosure.

### DETAILED DESCRIPTION

The techniques of this disclosure generally relate to medical device placement, including devices, systems and methods for placing needles, using a guide on a sterile ultrasound sheath.

In exemplary embodiments, the device described herein is configured to assist with any procedure where a needle is inserted into the body with real-time ultrasound guidance, e.g., for anesthesia (e.g., nerve blocks or pain procedures) or interventional radiology (e.g., cyst drainage or biopsy). In additional exemplary embodiments, the device described herein is configured to assist with plural such procedures.

In exemplary embodiments, the ultrasound device has software which depicts virtual needle paths corresponding to one or more of the needle guides. We note that such is particularly advantageous for in-plane targeting, with in exemplary embodiments an ultrasound display showing a virtual line for the in-plane path. For an out-of-plane path, in exemplary embodiments, the display shows a point of intersection midline on the display (a virtual needle intersection point), since the needle will cross at one point on the plane.

Furthermore, in exemplary embodiments, the ultrasound device has machine learning which allows it to identify the bony obstructions and to ensure the needle path is directed between the lamina.

In such procedures, there are two general steps: 1) establish the image of the target by placing the ultrasound against the patient; and 2) advance the needle to the target. As has been noted above, one challenge in the art involves coordination of components, e.g., maintaining proper visualization of the image as the needle is advanced.

Maintaining visualization during needle advancement can also be particularly challenging when the patient is obese. In this case, obtaining the image requires pushing several centimeters into the skin of the patient. The probe is touching the skin at the end of a tunnel; and there is no flat area of skin adjacent to the probe suitable for a needle entry site. Often an assistant is required to hold back the skin to allow access to the needle entry site. Even with assistance, this process can be difficult because the process of moving the skin away from the probe can cause loss of target visualization.

Exemplary embodiments describe a shell that at least partially houses the ultrasound probe. In exemplary embodiments, this shell is a rigid, sterile single use shell. Connected to or integral to the shell is a flange that moves the patient's skin away from the probe as it presses against the patient's skin. At the connection of the flange to the distal point of the shell, there are plural passageways for needles to be advanced at one or more fixed angles and/or at a continuum of angles, in either the in-plane or out-of-plane configuration relative to the ultrasound probe positioning.

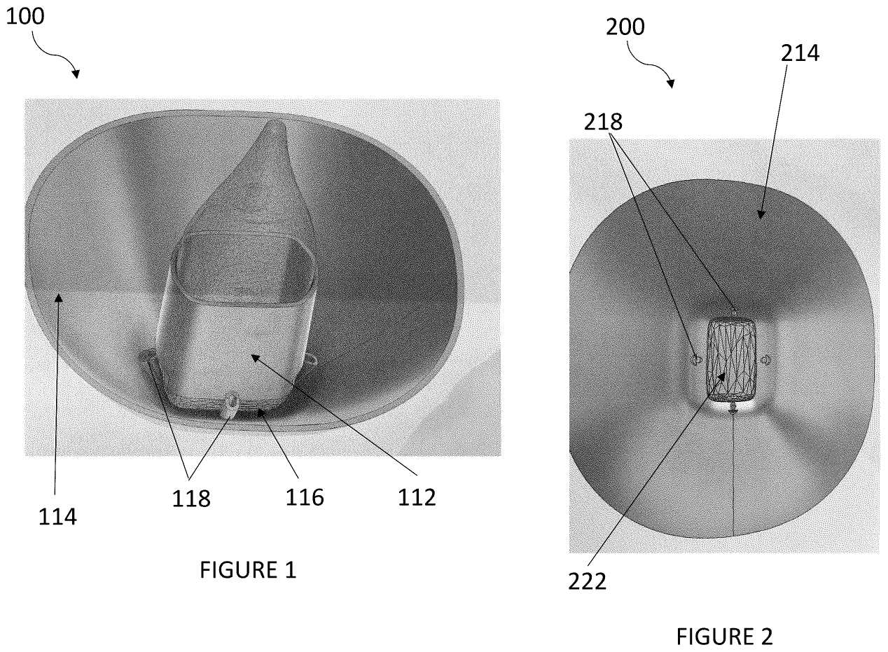

FIG. 1 illustrates a first exemplary ultrasound probe shell generally at 100, with the shell including an ultrasound probe sheath portion 112, a tissue deflecting flange 114, joined to the sheath portion at a distal end portion 116, and with plural needle guides 118 provided at the distal end portion.

An ultrasound probe is generally shown as a shaded portion 120 installed in the sheath 112. We note that a sterile barrier, such as a flexible sheath film or a telescoped tubular sheath of flexible film, may be provided around this proximal portion of the ultrasound sheath during installation. Additionally with reference to the bottom view of the shell in FIG. 2, a distal, flexible sterile material 222 can be provided integral to the distal end of the sheath 112 or as part of the ultrasound during insertion. In exemplary embodiments this is a thin film or membrane that maintains the sterile field but also does not interrupt visualization therethrough by the ultrasound head.

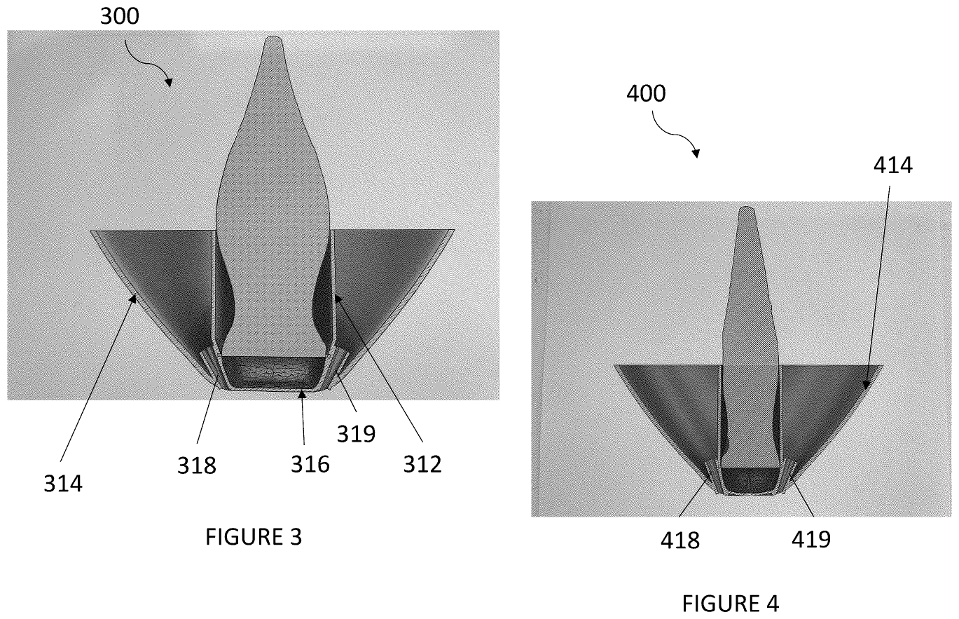

FIG. 3 is an exemplary block shell having sheath 312 and flange 314 components, as described by embodiments herein, shown in an in-plane cross section, with a continuum-angle needle guide on the right at 319 and fixed-angle needle guides 318 on the left. FIG. 4 is an exemplary block shell shown in an out-of-plane cross section, with a continuum-angle needle guide 418 on the left and fixed angle needle guides 419 on the right.

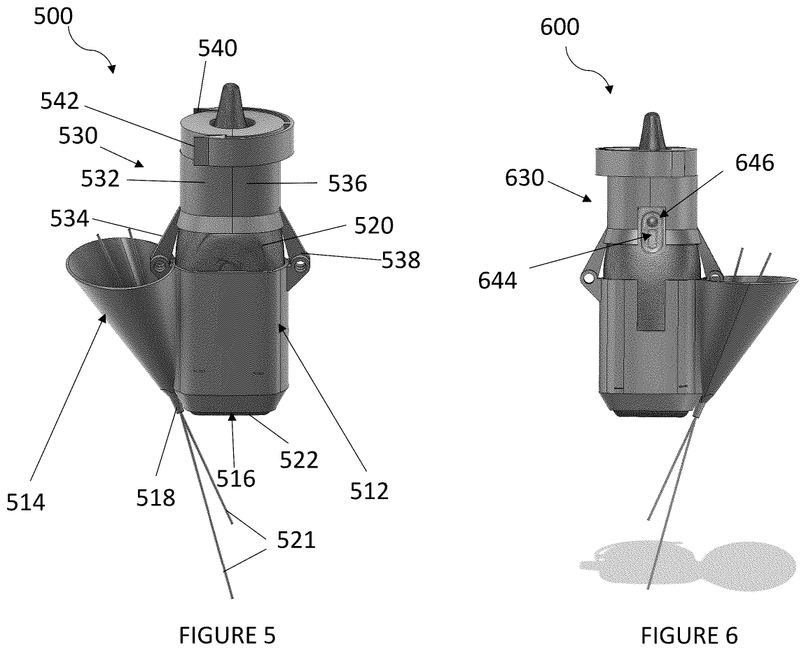

While FIG. 1 illustrates a first exemplary embodiment, with the flange 114 surrounding the ultrasound probe, other exemplary configuration are contemplated herein. For example, FIG. 5 illustrates a rear elevation view of a second exemplary embodiment generally at 500. This exemplary embodiment includes a sheath portion 512, joined or coupled with a flange portion 514. In this embodiment, the flange portion 514 is provided beside and connected or joined to the sheath portion 512. In such a way, targeted application of force to the tissue can be performed with the flange not surrounding the sheath 512. The flange to sheath interface also includes plural needle guides 518 at a distal end of the sheath (the paths of the needle guides are illustrated by the exemplary needles 521 extending therethrough.

Referring still to FIG. 5, the ultrasound device 520 is shown installed in the sheath 512 up to the distal end 516 (which again may utilize a thin membrane or film 522 between the ultrasound head and the patient). In this exemplary illustrated example, the ultrasound device is provided within a flexible tubular sheath that extends up into and past the exemplary proximal clamshell portion, shown generally at 530. We note that the exemplary flexible tubular sheath can also be provided with a proximal pull tab, to assist in maintaining a sterile field if the tubular sheath is being pulled up and over, e.g., a protruding wire for a wired ultrasound, or a proximal portion of a wireless ultrasound.

The exemplary clamshell portion 530 includes a first rigid clamshell piece 532 hingedly attached to the flange 514 and sheath 312 by a first hinged clamshell support 534 and a second rigid clamshell piece 536 hingedly attached to the sheath 312 by a second hinged clamshell support 538 opposite the first rigid clamshell support. As such, as the ultrasound device is installed, the clamshell portion 530 serves to hold the proximal end of the ultrasound in place to avoid shifting, e.g., using a catch or catches 540, 542 as part of the clamshell portion.

FIG. 6 illustrates a front elevation view of the device of FIG. 5 generally at 600, showing controls 644 for the ultrasound, beneath the flexible film or membrane around the ultrasound 620. This emphasizes that the proximal clamshell portion 630 (or e.g., the sheath portion) can have one or more cutouts 646 to accommodate for access to controls of the ultrasound device itself, as may be desired.

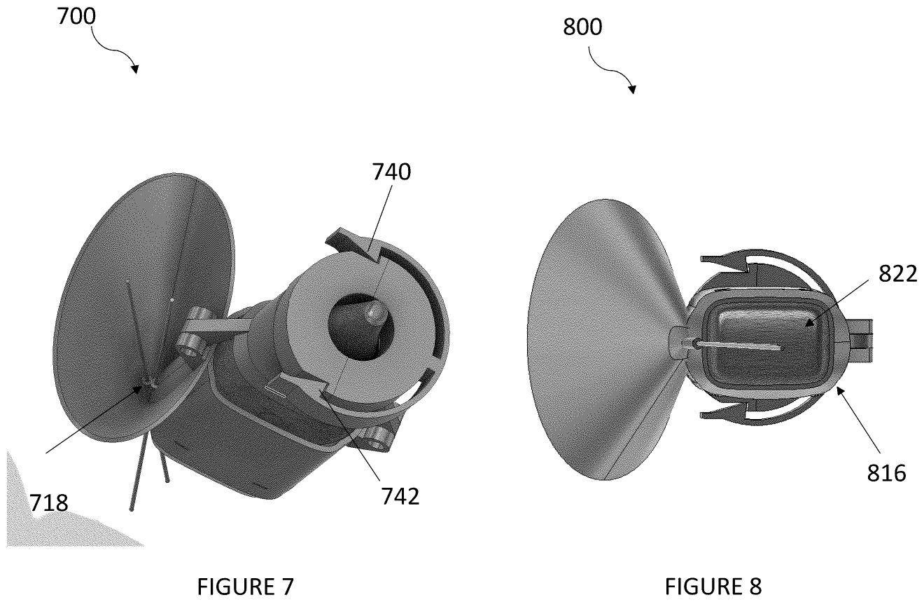

FIG. 7 illustrates a top persective view of the device of FIG. 5 generally at 700, showing the clamshell catches 740, 742 and the needle guides 718 in greater detail. FIG. 8 illustrates a bottom elevation view of the device of FIG. 5 better illustrating the exemplary flexible membrane or film 822 at the distal end 816 of the sheath.

In one exemplary aspect, a device is provided that facilitates single-operator procedures with real-time ultrasound guidance. The exemplary device allows the operator to place the needle with (e.g.) millimeter precision with one needle pass and complete the procedure without assistance, while maintaining a sterile field using a disposable shell.

In further exemplary embodiments, a system includes a plurality of devices produced/provided (e.g., in a kit or individually) in different versions, one for each of the commonly used commercial ultrasound probes and/or in different sizes or with different contouring for different patient categories. In further exemplary embodiments, software add-ons are provided for existing commercial ultrasound devices that further enhance the performance of the device.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A medical device, comprising:
a sterile medical device shell comprising:
a sheath configured to hold the distal end of an ultrasound device; and
a flange connected to the sheath and including plural needle guides at a distal end thereof and at a juncture with the sheath, such that the needle guides direct a needle into the path of an ultrasound image.

2. A device in accordance with claim 1, wherein the needle guides are each angled back to the path of an ultrasound probe beam directed by the sheath such that the guide trajectories of each intersect the beam at varying depths.

3. A device in accordance with claim 2, wherein the depths vary between 4 and 15 centimeters.

4. A device in accordance with claim 3, wherein the depths vary between 4 and 6 centimeters.

5. A device in accordance with claim 3, wherein the depths vary between 7 and 9 centimeters.

6. A device in accordance with claim 3, wherein the depths vary between 10 and 12 centimeters.

7. A device in accordance with claim 3, wherein the depths vary between 13 and 15 centimeters.

8. A device in accordance with claim 1, wherein the predetermined angle takes into account at least one patient parameter for that patient in order to achieve successful placement, including angle and depth of placement in target tissue.

9. A device in accordance with claim 8, wherein the at least one parameter is one or more of patient sex, age, height and weight.

10. A device in accordance with claim 1, wherein the predetermined angle is between 0 and 30 degrees.

11. A device in accordance with claim 10, wherein the predetermined angle is between 10 and 20 degrees.

12. A device in accordance with claim 1, further comprising a rigid proximal clamshell configured to engage a proximal end portion of the ultrasound.

13. A device in accordance with claim 12, wherein the clamshell portion includes a releasable catch configured to hold the clamshell together.

14. A device in accordance with claim 1, wherein the sheath includes a flexible film or membrane at a distal end portion thereof.

15. A device in accordance with claim 12, wherein a flexible film or membrane extends from the sheath at least partially through an interior aperture of the clamshell portion.

16. A device in accordance with claim 15, wherein the flexible film or membrane is configured as a flexible sheath that extends proximally such that it is configured to cover an ultrasound device provided therein.

17. A device in accordance with claim 16, wherein the flexible sheath is provided with a pull tab configured to slide a proximal portion of the flexible sheath up and around the ultrasound.

18. A device in accordance with claim 17, wherein the flexible sheath is configured with elasticity configured to tighten over an ultrasound cord extending proximally from the clamshell.

19. A device in accordance with claim 1, wherein the sheath or clamshell portion includes at least one cutout for ultrasound controls.

20. A device in accordance with claim 19, wherein the at least one cutout is provided with a flexible film or membrane configured to cover the ultrasound controls.
